(19) United States
(12) Patent Application Publication
(10) Pub. No.: US 2024/0166726 A1
(43) Pub. Date: May 23, 2024

(54) UNIVERSAL TARGETING STRATEGY TO INHIBIT REPLICATION OF ZIKV AND FLAVIVIRUSES

(71) Applicant: Sanford Burnham Prebys Medical Discovery Institute, La Jolla, CA (US)

(72) Inventors: Alexey V. TERSKIKH, Solana Beach, CA (US); Sergey A. SHIRYAEV, San Diego, CA (US)

(21) Appl. No.: 18/513,219

(22) Filed: Nov. 17, 2023

Related U.S. Application Data
(60) Provisional application No. 63/384,933, filed on Nov. 23, 2022.

(51) Int. Cl. C07K 16/10; A61K 31/14; A61K 47/55

(52) U.S. Cl. CPC C07K 16/10; A61K 31/14; A61K 47/55; C07K 2317/21; C07K 2317/24

(57) ABSTRACT

Methods and compositions related to treating flavivirus are described.

### CROSS REFERENCE

This Application claims the benefit of U.S. Provisional App. No. 63/384,933 filed on Nov. 23, 2022, which is incorporated by reference in its entirety herein.

### STATEMENT AS TO FEDERALLY SPONSORED RESEARCH

This invention was made with government support under R21 AI134581, and R01 NS105969 awarded by the National Institutes of Health. The government has certain rights in the invention.

### BACKGROUND

Flaviviruses infections, including Zika virus (ZIKV) infection, is a major global health threat. Currently, no effective countermeasures, such as drugs or vaccines, have been approved by the United States Food and Drug Administration for the treatment or prevention of ZIKV infection. Therefore, there is a need for developing therapies to prevent or treat flaviviruses infections.

### SUMMARY

In an aspect, the present disclosure provides a method of inhibiting replication of a flavivirus comprising a NS2B-NS3pro domain and a NS3hel domain, comprising: administering a chemical entity that interacts with the NS2B-NS3pro domain in an open conformation and the NS3hel domain.

In some embodiments, the chemical entity blocks a single strand RNA from interacting with the NS2B-NS3pro domain and the NS3hel domain. In some embodiments, the chemical entity comprises a NS2B-NS3pro binding moiety and a NS3hel binding moiety. In some embodiments, the chemical entity is a small molecule, a modified single strand RNA, a polynucleotide, a modified polypeptide, a fusion protein or an antibody. In some embodiments, the chemical entity is a small molecule. In some embodiments, the NS2B-NS3pro binding moiety and the NS3hel binding moiety of the small molecule are connected by a linker. In some embodiments, the chemical entity interacts with a positively charged groove on a surface of the NS3hel domain and two positively charged/polar forks of the NS2B-NS3pro domain. In some embodiments, the chemical entity is an antibody. In some embodiments, the antibody is a human antibody or humanized antibody. In some embodiments, the chemical entity is a fusion protein. In some embodiments, the fusion protein comprises human or humanized regions. In some embodiments, the flavivirus is Zika virus (ZIKV), West Nile virus (WNV), dengue virus (DENV serotypes 1-4), Japanese encephalitis virus, hepatitis C virus (HCV), or tick-borne encephalitis virus.

In another aspect, the present disclosure provides a method of inhibiting replication of a flavivirus, comprising: administering a chemical entity or a biological entity that simultaneously interferes with single strand RNA binding and protease catalytic activity during viral propagation of the flavivirus.

In some embodiments, the chemical entity or the biological entity is an anti-NS2B-NS3pro inhibitor. In some embodiments, the anti-NS2B-NS3pro inhibitor targets one or more pockets of a NS2B-NS3pro domain when the NS2B-NS3pro domain is in a super-open conformation. In some embodiments, the anti-NS2B-NS3pro inhibitor targets the cavities formed by the closely opposed ZIKV NS3pro and NS3hel domains thus inhibiting the alignment of the NS3pro and NS3hel domains.

### INCORPORATION BY REFERENCE

All publications, patents, and patent applications mentioned in this specification are herein incorporated by reference to the same extent as if each individual publication, patent, or patent application was specifically and individually indicated to be incorporated by reference. DUAL FUNCTION OF ZIKA VIRUS NS2B-NS3 PROTEASE, Sergey A. Shiryaev, Anton Cheltsov, Robert C. Liddington, Alexey V. Terskikh, available at: www.biorxiv.org/content/10.1101/2021.11.28.470275v1 (hereinafter, “the biorxiv article”), and DUAL FUNCTION OF ZIKA VIRUS NS2B-NS3 PROTEASE, Sergey A. Shiryaev, Anton Cheltsov, Robert C. Liddington, Alexey V. Terskikh, available at: www.biorxiv.org/content/10.1101/2021.11.28.470275 (hereinafter “the biorxiv article updated version”), all of which are incorporated by reference herein in their entirety.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein) of which:

FIG. 1 illustrates ZIKV polyprotein composition and processing by viral and host cell proteases. Proteolytic cleavage sites are indicated by arrows.

FIG. 2 illustrates the transitional equilibrium between closed, open, and super-open conformations of ZIKV NS2B-NS3pro.

FIG. 3 depicts an exemplary Western blot analysis of the schematics of NS2B-NS3pro constructs provided herein.

FIGS. 4A-4D illustrate that ZIKV NS2B-NS3pro binds ssRNA and ssDNA.

FIGS. 5A-5B illustrate the structural and functional alterations in ZIKV NS2B-NS3pro that impact ssRNA binding.

FIGS. 6A-6B illustrate that binding to RNA inhibits the proteolytic activity of NS2B-NS3pro.

FIG. 7 is a bar graph depicting the inhibition of ssRNA binding by allosteric inhibitors of ZIKV NS2B-NS3pro.

FIG. 8 illustrates the structures of ZIKV NS2B-NS3pro co-crystallized with NSC86314 (super-open) and predicted by SWISSDOC (open).

FIG. 9 illustrates 3 confirmations of ZIKV NS2B-NS3pro, with the open conformation poised to bind RNA

FIGS. 10A-10B illustrate modeled and crystalized structures of ZIKV NS3hel with RNA fragments.

FIGS. 11A-11D illustrate the modeling of ZIKV NS3pro-NS3hel mutual orientations.

FIGS. 12A-12C illustrate the models of the RNA-NS2B-NS3pro-NS3hel complex.

FIGS. 13A-13B illustrate the volumes and position of the cavities at the NS3pro and NS3hel interface.

FIGS. 14A-14E illustrate a “reverse inchworm” mechanism of the helicase-protease structure-activity cycle for ZIKV NS2B-NS3 complex.

FIGS. 15A-15B illustrate that negatively charged forks, a novel druggable site, are conserved across flaviviruses. Drugs comprise antiviral activities against flavirviruses can be designed to target this region.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Provided herein are methods of inhibiting replication of flaviviruses. In some embodiments, the methods disclosed herein are effective at inhibiting replication of Zika virus (ZIKV). In some embodiments, the methods disclosed herein are effective at inhibiting the serine protease of a flavivirus, such as the NS2B-NS3pro. In some embodiments, the methods disclosed herein comprise inhibiting the NS2B-NS3pro of a flavivirus by providing an entity to inhibit both RNA-binding capacity and proteolytic activity of the NS2B-NS3pro of a flavivirus. In some embodiments, the methods described herein can be used by those skilled in the art to screen compound libraries (physically and/or virtually) for chemical entities that inhibit the alignment of the NS3pro and NS3hel domains during flavivirus replication to prevent viral propagation thereof.

Flaviviruses may include, for example. Zika Virus (ZIKV) Zika virus (ZIKV), West Nile virus (WNV), dengue virus (DENV scrotypes 1-4), Japanese encephalitis virus, hepatitis C virus (HCV), or tick-borne encephalitis virus. Zika virus (ZIKV) serine protease is indispensable for viral polyprotein processing and replication and comprises an NS2B polypeptide that associates with a proteolytic N-terminal fragment of NS3 polypeptide (NS3pro) to form NS2B-NS3pro. NS2B-NS3pro is s likely to be a universal feature of Flaviviridae, given the high level of homology between NS3 protease-helicase proteins in this family. NS2B-NS3pro constructs may alternate between several states, closed, open, and super open. In open and/or super open confirmations, the NS3hel domain provides a positively charged surface contiguous with the NS3 helicase domain. In some aspects, when the ZIKV protease adopts the “open” conformation and/or “super-open conformation” two positively charged “forks” may be present and interference with these forks may be interfered with by a single chemical or biological entity that could then prevent Zika virus and/or all Flaviviridae propagation. During viral propagation, the N-terminal proteolytic (NS3pro) and C-terminal helicase (NS3 helicase) domains function within the constraints of a single NS3 polypeptide. NS2B-NS3pro is responsible for autoproteolytic cleavage of junction regions between NS2B/NS3 and NS3/NS4A proteins bordering NS3 protein during viral polyprotein processing. After this step NS3 protein is associated with the ER membrane via NS2B cofactor (4 transmembrane domains) which interacts with NS3pro domain and NS4A cofactor (2 transmembrane domains) which interacts with NS3 helicase domain. Based on our experimental, structural, and modeling studies, it is likely that NS3pro not only has proteolytic activity but also binds one ssRNA strand, thereby facilitating the unwinding of dsRNA generated by viral NS5 RNA-dependent RNA polymerase. We propose the existence of a dynamic equilibrium between the closed-open-super-open conformations of NS2B-NS3pro. The transition between conformations depends on the coordinated structural rearrangement of the C-terminal tail of NS3pro and the linker between NS2B-NS3pro and NS3 helicase, resulting in progressive loss of interactions between NS3pro and NS2B

In support of our findings, several studies have pointed to the importance of the flaviviral NS3 protease domain for proper functioning of NS3 helicase. The presence of the protease domain within full-length HCV NS3 protease-helicase constructs was shown to significantly enhance direct and functional RNA binding to NS3 helicase. Additionally, the presence of the NS3 protease domain contributed substantially to helicase translocation stepping efficiency by increasing the efficiency up to 93% per ATP hydrolysis event relative to 20% for the single NS3 helicase construct. Absence of the NS3 protease domain results in malfunctioning of NS3 helicase directional movement and the appearance of two opposing activities characterized by an ATP-dependent steady state between RNA unwinding and RNA annealing processes.

The linker between the NS3 protease and helicase domains has been demonstrated to play a critical role in their activities in the structurally and functionally similar DENV2 NS2BNS3pro. Mutations in the linker affecting its flexibility and length were crucial for the ATPase and helicase activities and also led to significant reductions in viral genomic RNA synthesis. Given the high negative charge of these 12 aa (171EEETPVECFEPS182), we propose that the linker between NS3 protease and helicase domains competes with ssRNA for binding to NS3pro. This may provide an explanation for the lack of ssRNA binding by the NS2B-NS3pro-long construct shown here.

In crystal structures, the ZIKV protease-helicase linker is disordered (PDB IDs 5TFN, 5TFO, 5T1V and 6UM3). However, in the closed and open structures of WNV and DENV2 proteases, the linkers are associated with a conserved region of the protease domain. We suggest that protease-helicase linker of ZIKV could dynamically interact with the protease domain, and its negative charge might weaken ssRNA binding (e.g. NS2B-NS3pro-long).

Published structures of ZIKV protease in the super-open state show that Ser160 is the last ordered NS3 residue, whereas this is Gly168 for proteases in the closed and open conformations.

The dissociation of NS2B from NS3pro during the transition from closed to open to super open conformations changes the isoelectric point (pI) of the NS2B-NS3pro complex. Note that the progressive dissociation of NS2B from NS3pro removes the regions containing negatively charged residues in a conformation-dependent fashion, leading to an increased pI of the NS2BNS3pro complex that should facilitate ssRNA binding.

Recent crystal structure of ZIKV NS2B-NS3pro in the super-open conformation with a small molecule compound occupying a new pocket (7M1V), suggests it may be feasible to specifically target the super-open conformation of NS2B-NS3pro. Molecular probes targeting the super-open conformation will help to elucidate the effects of the different NS2B-NS3 conformational states on the protease and the helicase activities.

Based on the model of dynamic switching between closed-open-super-open conformations, we propose that NS2B-NS3pro dynamically cycles between binding and releasing ssRNA, perhaps assisting NS3 helicase in pulling and unwinding dsRNA. In support of this proposal, the HCV NS3 protease has been shown to be important for directional movement of the NS3 helicase and translocation stepping efficiency in ATP hydrolysis during the viral dsRNA unwinding process.

The C-terminal regions of NS2B cofactors from various flaviviruses contain a conserved negative charge, which may explain why the productive binding of this fragment by protease in a closed state inhibits binding to negatively charged nucleotides. In the crystal structures of flavivirus proteases in the open state, the negatively charged fragment of NS2B is either disordered or loosely bound to NS3pro surface. Thus, differences in the location of the negatively charged NS2B fragment between the open and closed states is consistent with the preferred binding of ssRNA to the open conformation. Ongoing studies, including modeling of ssRNA binding to ZIKV NS2B-NS3, provide insights into the dynamic interactions of the protease and helicase domains.

The binding of ssRNA to ZIKV NS2B-NS3pro in an open conformation provides new insights into the functional cycle of ZIKV RNA processing and viral replication. The appearance of pockets in NS2B-NS3pro accessible only in the open and super-open states provides for those skilled in the art to design novel allosteric inhibitors that simultaneously affect ssRNA binding and protease catalytic activity. This information facilitates the development of new approaches for the design of allosteric inhibitors not only of ZIKV but also of other members of the Flaviviridae family, given their high (>70%) primary sequence homology and high structural similarity.

In flaviviruses the proteolytic activity of NS2B-NS3pro and the helicase activity of NS3hel are constrained within a single NS3 polypeptide. Extensive structural and functional studies conclusively demonstrated a strong interdependence in the enzymatic activities of the protease and helicase domains of flaviviral NS3 protein. However, multiple studies have pointed to the critical role of the flaviviral NS3pro domain for proper functioning of NS3 helicase. The protease domain of HCV NS3 protein plays significant role in stepping efficiency of NS3hel by increasing the efficiency of the coupling construct NS3-NS4A up to 93% per each ATP hydrolysis event relatively to 20% for single NS3 helicase construct. The presence of the NS3pro domain within full-length HCV NS3 protease-helicase constructs was shown to significantly enhance RNA binding to NS3 helicase. Absence of the DENV2 NS3pro domain results in malfunctioning of DENV2 NS3 helicase directional movement and the appearance of two opposing activities characterized by an ATP-dependent steady state between RNA unwinding and RNA annealing processes. The DENV4 virus full-length NS3 protein displayed 10-fold higher ATP affinity than the isolated helicase domain. Moreover, for DENV2 virus NS3 helicase-protease construct the RNA unwinding activity was about 30-fold higher than that of the NS3hel construct representing only helicase domain. Also, a higher rate of RNA unwinding was reported for Kunjin virus (KUNV) full-length NS3 protein when compared to construct continuing only NS3hel domain. Despite the robust evidence that NS3pro is important for the helicase function, the exact mechanism of the NS2B-NS3pro involvement in NS3 helicase activity was previously unknown.

Herein, a novel “reverse inchworm” model for a tightly intertwined NS2B-NS3 helicase-protease machinery of ZIKV and flaviviruses in general is proposed. The key element of this model is a “catch and release” cycle where NS2B-NS3pro binds RNA in the open conformation and releases RNA in the super-open conformation. The binding of RNA is mediated via two fork-like structures present only in the open conformation of NS2B-NS3. The amino acid sequences and 3D arrangements of these positively charged structures are highly conserved across flaviviruses suggesting a universal nature of the proposed mechanism.

The protease and helicase activities of NS2B-NS3 complex are mutually exclusive. A model for spatial separation of these activities has been proposed where organelle-like alterations of ER membranes called replication factories (RF) were associated with ZIKV/flaviviral infections. The detailed architecture of RFs has been reconstructed using electron tomography. Replication Factories consist of several sub-structures including vesicle packets (VP) and virus bags (newly assembled virions) which are morphologically distinct. According to this model, NS2B-NS3 complex will function mostly as a helicase within VPs and mostly as a protease outside VPs since ribosome clusters indicative of translation areas were visualized adjacent to but outside of VP structures. However, how the intact ˜11 kB viral RNA is transported out of VPs and loaded on the ribosomes is unclear. A model of co-transcriptional translation where RNA is associated with ribosomes right after the release from NS2B-NS3 is rather appealing.

ZIKV NS3 is likely a canonical RNA helicase but previously observed a very limit processivity of recombinant NS3 polypeptide that was capable of unwinding no longer than 18 bp RNA duplex. Curiously, addition of NS5 increase the speed, but doesn't change the processivity (max 18 bp RNA duplex) of NS3. Note that this recombinant NS3 helicase was lacking NS2B peptide, thus could not adopt proteolytically active closed conformation of ZIKV NS2B-NS3pro (PDB ID 5LC0). The crystal structures of NS2B-NS3 protease in super-open conformation (PDB IDs 5TFN, 5TFO, 6UM3, and 7M1V) and our modeling suggest that in the absence of NS2B cofactor NS3pro will adopt an open conformation capable of RNA binding. This is consistent with the observation that NS3hel alone (truncated polypeptide lacking NS3pro domain) has no helicase activity whatsoever. Indeed, regardless of the exact mechanism of action all DNA or RNA helicases have 2 binding sites to enable enzyme processivity. According to our reverse inchworm model, without NS2B cofactor NS3pro is unable to switch to super-open conformation that forces RNA dissociation thus limiting the processivity of NS3hel by the maximal elongation of 12 aa linker. We predict that with the addition of NS2B cofactor, NS2B-NS3 complex will exhibit unlimited processivity needed to dissociate the entire ZIKV dsRNA of ˜11 kb.

The 12 aa linker between the NS3pro and NS3hel domains was shown to be important critical for the protease and helicase activities in the structurally and functionally similar DENV2 NS2B-NS3pro. Mutations in the linker affecting its flexibility and length were crucial for the ATPase and helicase activities and also led to significant reductions in viral genomic RNA synthesis. The linker is negatively charged due to 5 glutamic acid residues spread along the sequence shown in the biorxiv article at p. 8, paragraph 2; and p. 13, paragraph 4. In crystal structures, the ZIKV protease-helicase linker is disordered (PDB IDs 5TFN, 5TFO, 5T1V and 6UM3). However, in the closed and open structures of WNV and DENV2 proteases, the linkers are associated with a conserved region of the protease domain. We propose that this flexible negatively charged peptide competes with RNA for binding to NS2B-NS3pro. This may provide an explanation for the observed lack of RNA binding by the NS2B-NS3pro-long construct that includes the 12 aa linker.

Finally, the reverse inchworm model posits that NS3hel hands over the threaded RNA strand to the NS2B-NS3pro. This process should be most efficient when the NS3hel positively charged groove is contiguous with the positively charged fork-like structures in the open conformation of NS2B-NS3pro. The volumes of the cavities formed by closely opposed ZIKV NS3pro and NS3hel domains are consistent with the possibility to design small molecule that would bind on one or both sides thus interfering with this arrangement. Such opposition of NS3pro and NS3hel domains should be important for the most efficient RNA binding by NS2B-Ns3pro. As such, targeting the interface of the specific alignment of NS3pro and NS3hel domains proposed herein with small molecules could likely be a universal approach targeting the replication of ZIKV and flaviviruses in general.

### EXAMPLES

The following examples are provided to further illustrate some embodiments of the present disclosure, but are not intended to limit the scope of the disclosure; it will be understood by their exemplary nature that other procedures, methodologies, or techniques known to those skilled in the art may alternatively be used.

### Materials and Methods

Reagents. Routine laboratory reagents were purchased unless indicated otherwise. Oligonucleotides were synthesized by Integrated DNA Technologies.

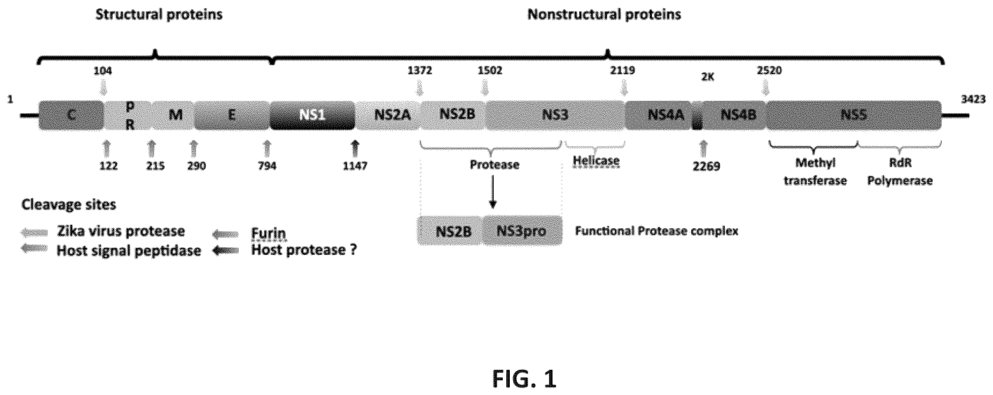

Cloning, expression, and purification of ZIKV NS2B-NS3pro constructs. FIG. 1 illustrates ZIKV polyprotein composition and processing by viral and host cell proteases. Positions of cleavage sites for host and viral proteases at the junctions between individual viral proteins are indicated by arrows. Supplementary FIG. 1C in the biorxiv article shows the NS2B-NS3pro cleavage sequences in flaviviral polyproteins. Cleavage sites in the capsid protein C and at the NS2A/NS2B, NS2B/NS3, NS3/NS4A, NS4A/NS4B, and NS4B/NS5 boundaries are shown. ZIKV, Zika (GenBank AMB37295); WNV, West Nile virus (GenBank P06935); JEV, Japanese encephalitis (GenBank P19110); YFV, yellow fever (GenBank P19901); DENV1-4, dengue serotypes 1-4 (GenBank P33478, P29990, P27915, and P09866, respectively).

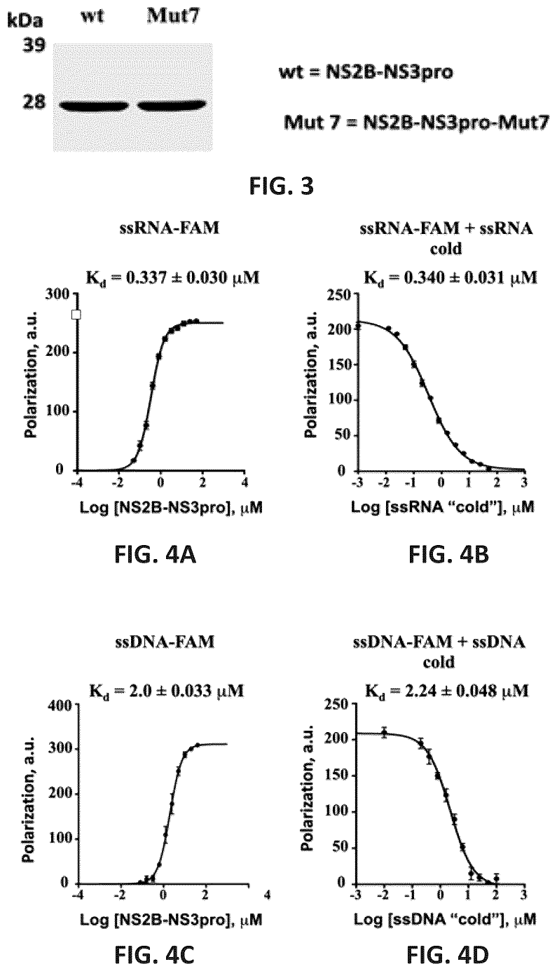

DNA sequences for the ZIKV constructs were synthesized and codon-optimized for efficient transcription in E. coli. As depicted in Supplementary FIG. 1A in the biorxiv article, the constructs were designed as single-chain two-component products lacking the hydrophobic transmembrane domain of the NS2B cofactor. To achieve this, the hydrophilic cytoplasmic portion of NS2B (residues 48-94) was linked to the NS3 protease domain via a 9-residue linker L shown in the description of Supplementary FIG. 1A in the biorxiv article . All constructs were N-terminally fused with GST protein or HisTag for purification. FIG. 3 illustrates the Western blot analysis of purified wild-type and Mut7 NS2B-NS3pro proteins. To block the proteolytic activity of the construct, where indicated, the catalytic Ser135 was substituted with Ala to give the inactive Ser135Ala construct.

ZIKV NS2B-NS3pro recombinant constructs with N-terminal His tag were used to transform competent E. coli BL21 (DE3) Codon Plus cells. Transformed cells were grown at 30° C. in LB broth containing carbenicillin (0.1 mg/ml). Protein production was induced with 0.6 mM IPTG for 16 h at 18° C. Cells were collected by centrifugation at 5000 g at 4° C., and the cell pellet was resuspended in 20 mM Tris-HCl buffer, pH 8.0, containing 150 mM NaCl (TBS), and sonicated (eight 30 s pulses at 30 s intervals) on ice. The sample was then centrifuged at 40,000 g for 30 min at 4° C. and the constructs were purified from the supernatant fraction using Ni-NTA Sepharose equilibrated with TBS containing 1 M NaCl. Impurities were removed by washing with the same buffer supplemented with 35 mM imidazole, and the column was equilibrated with standard TBS. The beads were co-incubated with thrombin to cleave the His tag and release NS2B-NS3pro.

Fractions containing recombinant protein were combined and purified by gel filtration on a GE S200 26/60 column equilibrated with standard TBS. Fractions containing ZIKV NS2B-NS3pro were concentrated to approximately 10 mg/ml using 10 kDa-cutoff concentrators, and then flash frozen in small aliquots and stored at −80° C. Purity of the material was checked by SDS-PAGE on 12% NuPAGE-MOPS gels, followed by Coomassie staining.

A series of constructs (Mut3, Mut5, and Mut6) that forced ZIKV NS2B-NS3pro to adopt the “super-open” conformation was generated using the wild-type construct as a template. In the Mut3 construct, all three Cys residues in the original sequence were substituted with Ser residues (Cys80Ser, Cys143Ser, and Cys178Ser). In the construct with the “super-open” conformation only (Mut5), two additional Cys residues were inserted into the Mut3 construct (Ala88Cys and Lys157Cys). To obtain a catalytically inactive mutant with the “super-open” conformation (Mut6), an additional Ser135Ala was introduced into the Mut5 construct. For crystallization purposes, a Mut7 construct containing Leu30Thr and Leu31Ser mutations in the Mut5 sequence was created. Mut7 was designed to minimize ZIKV protease dimerization during crystallization.

The DNA constructs were cloned into pGEX6P1 plasmid using BamH1 and EcoR1 cleavage sites, resulting in fusion of a GST tag at the N-terminus of the NS2B cofactor. E. coli were transformed with the individual recombinant constructs, and protein production was induced and the cells were disrupted as described above. The supernatant fraction containing the GST-tagged constructs were loaded onto Protino Glutathione Agarose 4B beads and impurities were removed by washing with TBS. To cleave the GST tag from the viral protease, the beads were co-incubated with 3C protease. The NS2B-NS3pro constructs were then additionally purified by gel filtration on an S200 26/60 column equilibrated with TBS. Purity of the purified construct was analyzed using SDS-PAGE followed by Coomassie staining. Purified protease was concentrated to approximately 10 mg/ml using 10 kDa-cutoff concentrators, flash frozen in small aliquots, and stored at −80° C. To isolate NS2B-NS3pro complexed with aprotinin or WRPK3, the NS2B-NS3pro constructs were incubated with each inhibitor at an equimolar ratio and the protein-inhibitor complexes were purified by gel filtration on a S200 Superdex column.

Fluorescent proteinase activity assay and IC50 determination. The peptide cleavage activity assays with purified ZIKV NS2B-NS3pro polypeptides were performed in 0.2 ml TBS containing 20% glycerol and 0.005% Brij 35 containing 20 μM of the cleavage peptide pyroglutamic acid Pyr-Arg-Thr-Lys-Arg-7-amino-4-methylcoumarin (Pyr-RTKR-AMC) and 10 nM enzyme. The reaction velocity was monitored continuously at λex=360 nm and λem=465 nm on a Tecan fluorescence spectrophotometer (Männedorf, Switzerland). To determine the IC50 values of the inhibitory compounds, ZIKV NS2B-NS3pro constructs (20 nM) were preincubated for 30 min at 20° C. with various concentrations of compounds in 0.1 ml TBS containing 20% glycerol and 0.005% Brij 35. Pyr-RTKR-AMC substrate (20 μM) was then added in 0.1 ml of the same buffer. IC50 values were calculated by determining the compound concentration required to obtain 50% of the maximal inhibition of NS2B-NS3pro activity against Pyr-RTKR-AMC. GraphPad Prism was used as fitting software. All assays were performed in triplicate in 96-well plates.

Fluorescent proteinase activity assay and IC50 determination. The peptide cleavage activity assays with purified ZIKV NS2B-NS3pro polypeptides were performed in 0.2 ml TBS containing 20% glycerol and 0.005% Brij 35 containing 20 μM of the cleavage peptide pyroglutamic acid Pyr-Arg-Thr-Lys-Arg-7-amino-4-methylcoumarin (Pyr-RTKR-AMC) and 10 nM enzyme. The reaction velocity was monitored continuously at λex=360 nm and λem=465 nm on a Tecan fluorescence spectrophotometer (Männedorf, Switzerland). To determine the IC50 values of the inhibitory compounds, ZIKV NS2B-NS3pro constructs (20 nM) were preincubated for 30 min at 20° C. with various concentrations of compounds in 0.1 ml TBS containing 20% glycerol and 0.005% Brij 35. Pyr-RTKR-AMC substrate (20 μM) was then added in 0.1 ml of the same buffer. IC50 values were calculated by determining the compound concentration required to obtain 50% of the maximal inhibition of NS2B-NS3pro activity against Pyr-RTKR-AMC. GraphPad Prism was used as fitting software. All assays were performed in triplicate in 96-well plates.

Fluorescence polarization assay of RNA/ssDNA binding to ZIKV NS2B-NS3 protease. Binding between purified recombinant ZIKV NS2B-NS3pro constructs and either RNA or ssDNA was assessed using a fluorescence polarization (FP) assay conducted in 0.1 ml of TBS containing 1 mM MgCl2 at 25° C. for 1 h. Samples of 10 nM 6-carboxyfluorescein-labeled 20-base RNA (poly-rA or -rU) or ssDNA (poly-dA or -dT) oligonucleotides were incubated with 50 nM to 50 μM of purified NS2B-NS3pro constructs. Polarization was monitored on a Bruker Daltonics fluorescence spectrophotometer. Kd values for RNA and ssDNA binding were calculated by determining the construct concentration needed to reach 50% polarization for 3′-fluorescein amidite-labeled RNA/DNA. All assays were performed in triplicate in 96-well plates. GraphPad Prism was used as fitting software.

Molecular modeling. All structural modeling presented here used the FFAS sequence alignment server for finding structural templates in Protein Data Bank and the MODELLER program for creating homology models. For building structural model for the full protein sequence, e.g., ZIKV NS2B co-factor protein, RoseTTAFold program was employed, which uses deep learning approach for quick structure prediction. Volumes and position of the cavities at the NS3pro and NS3hel interface were calculated using POVME3.0 program.

### Example 1: Modeling of NS2B-NS3pro Open Conformation

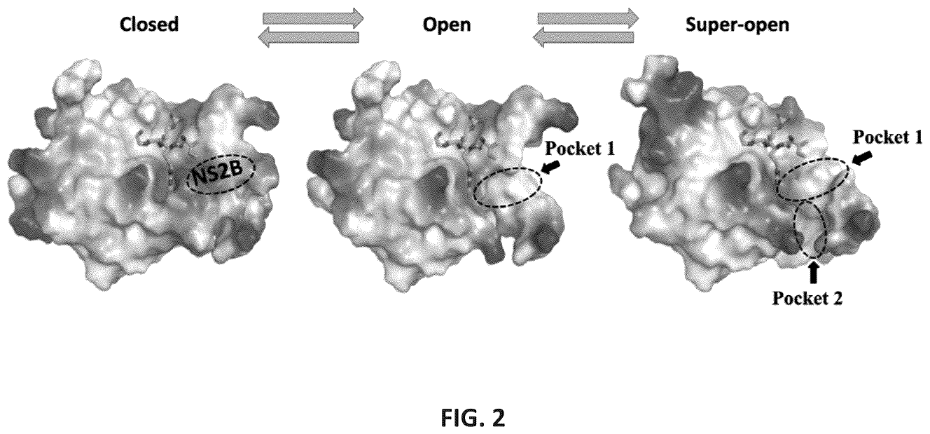

The structures of DENV and WNV NS2B-NS3pro crystalized in the open conformation (PDB IDs: 2FOM and 2GGV) were used to model ZIKV NS2B-NS3pro with the help of FFAS, MODELLER, and RoseTTAFold packages. Additional insights were obtained by modeling the removal of the C-terminal tail of the NS2B cofactor from a hydrophobic cleft of ZIKV NS2B-NS3pro in the closed conformation. Note that compared to the open conformation, the super-open conformation induces further dissociation of NS2B from NS3pro and a refolding of NS3pro C-terminal residues, which substantially affects overall fold of NS3pro. Given the apparent continuum of these structural changes, NS2B-NS3pro can dynamically switch between closed, open, and super-open conformations (FIG. 2). FIG. 2 illustrates the transitional equilibrium between closed, open, and super-open conformations of ZIKV NS2B-NS3pro with pockets potentially involved in binding of RNA or allosteric inhibitors indicated. A peptide-based substrate (ball and stick model) in the protease active center is modeled on the related structure of WNVpro+aprotinin (PDB ID: 2IJO). Orientation of the enzymes is chosen in such a way that RLLG loop interacting with the membrane surface is facing down.

### Example 2: ZIKV NS2B-NS3pro Polypeptide Binds RNA, which Inhibits Its Proteolytic Activity

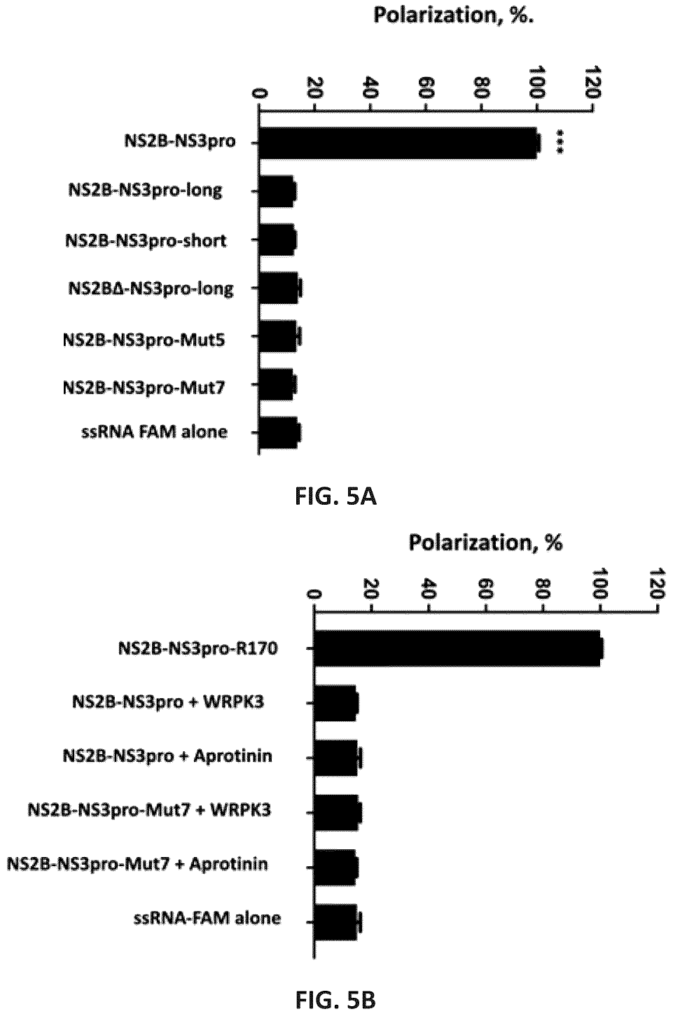

To investigate RNA and ssDNA binding to ZIKV NS2B-NS3pro polypeptide, classical fluorescence polarization (FP) assays were performed in which FAM-labeled RNA or ssDNA was incubated with NS2B-NS3pro, and binding was detected by a change (increase) in FP. As depicted in FIG. 4A, wild-type proteolytically competent NS2B-NS3pro, which is capable of adopting closed, open, or super-open conformations, bound robustly to FAM-labeled ssRNA 20 poly-rU (e.g., ssRNA-FAM). The results of direct binding assays and competition assays performed in the presence of unlabeled ssRNA 20 poly-U (e.g., ssRNA cold), shown in FIG. 4B, were in good agreement. Both binding assays gave Kd values of about 0.34 μM. An increase in FP signal was also obtained upon incubation of NS2B-NS3pro with FAM-labeled ssDNA 20 poly-dT, as depicted in FIG. 4C, indicating binding of NS2B-NS3pro to ssDNA, which can be inhibited by addition of unlabeled ssDNA shown in FIG. 4D. Both the direct binding and competition assays yielded Kd values for ssDNA that was ˜10-fold lower than the Kd for binding of RNA (˜2.0 M vs 0.3 μM). The demonstration that unlabeled RNA and ssDNA efficiently competed with the FAM-labeled probes with similar kinetics (FIGS. 4B and 4D) confirmed the specificity of binding. To investigate RNA binding to ZIKV NS2B-NS3pro polypeptide in more detail, a set of constructs aimed at probing some key structural and conformational requirements were generated (illustrated in Supplementary FIG. 1A in the biorxiv article). NS2B-NS3pro-longcontained 182 N-terminal residues of NS3 (NS2B-NS3-long) and a 12-aa linker region shown in the biorxiv article at p. 8, paragraph 2; and p. 13, paragraph 4 connecting the NS3pro domain with the NS3 helicase domain. NS2B-NS3pro-long can adopt the closed, open, and super-open conformations, and was crystallized in the super-open conformation (PDB ID 5TFN). As depicted in FIG. 5A, NS2B-NS3-long incubation with ssRNA FAM or ssDNA FAM did not increase the FP signal, indicating a lack of RNA or DNA binding.

The truncated NS2B-NS3-short construct containing 160 N-terminal residues of NS3 (NS2B-NS3- short) can only adopt the super-open conformation due to the lack of aa 161-170, which are necessary to form the closed or open conformations (PDB 5LC0). FP assays indicated that NS2B-NS3-short also failed to bind to ssRNA or ssDNA, as depicted in FIG. 5A, indicating a requirement for aa 161-170 in NS3pro to support RNA binding.

NS2B-NS3pro-Mut5 and Mut7 constructs were designed to be stabilized in the super-open conformation via a disulfide bond between two cysteines introduced into the NS3 sequence. Based on the solved structures of Mut5 and Mut7, both mutants can adopt the super-open conformation (PDB IDs 6UM3, 7MIV). As depicted in FIG. 5A, neither of these mutants was able to bind RNA, suggesting that the super-open conformation can be refractory to RNA binding. The NS2BA-NS3 pro construct contained a truncated NS2B (NS2BA) and lacked the sequence disclosed in the biorxiv article on p. 9, paragraph 3, which precludes the protein from adopting a closed conformation and forming a proteolytically active complex with NS3. The NS2BA-NS3-long construct was also unable to bind RNA (FIG. 5A).

Full-length NS2B-NS3pro polypeptide can efficiently bind RNA and the tested structural modifications, particularly those that lock NS2B-NS3pro in the super-open conformation, can eliminate RNA binding. The C-terminal region of NS3pro and the linker between NS3pro and NS3 helicase can modulate RNA binding.

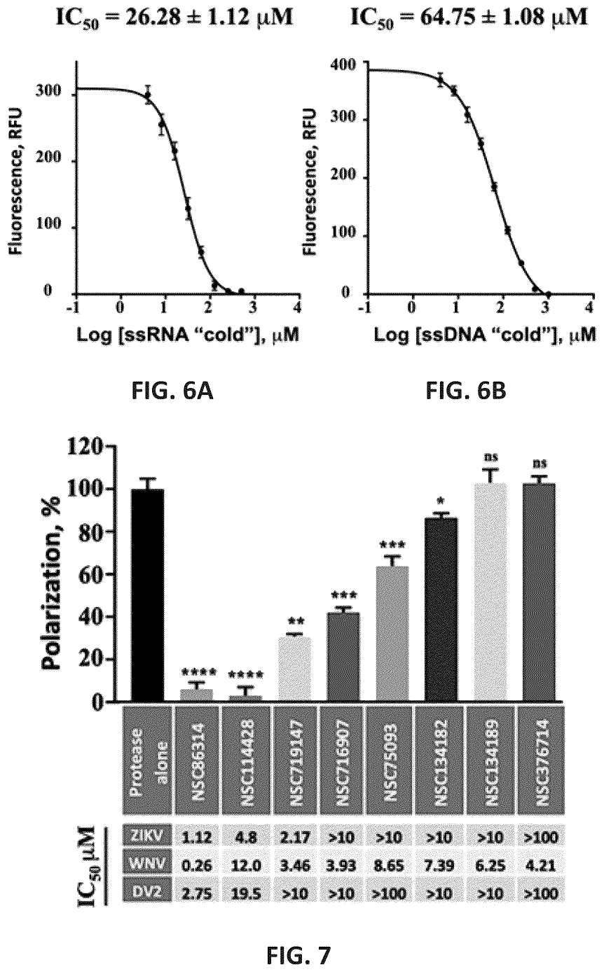

Fluorogenic substrate cleavage assays were performed to test whether binding of RNA interferes with the proteolytic activity of NS2B-NS3pro. As depicted in FIGS. 6A-6B, binding of ssRNA or ssDNA inhibited the proteolytic activity of ZIKV NS2B-NS3 protease with IC50 values of 26.28±1.12 μM for ssRNA and 64.75±1.08 μM for ssDNA. Thus binding of RNA or DNA to ZIKV NS2B-NS3 can inhibit its proteolytic activity, which can be due to stabilization of the polypeptide in the open, proteolytically inactive conformation.

### Example 3: NS2B-NS3pro Substrate-Mimicking Inhibitors Compete with RNA Binding

To determine whether RNA can bind to ZIKV NS2B-NS3pro in the closed, proteolytically active state, two substrate-mimicking inhibitors were used to induce the closed confirmation: aprotinin, a 70-aa serine protease inhibitor; and WRPK3, a synthetic peptide inhibitor that covalently binds to the NS2B-NS3pro active center . Previous studies demonstrated that these inhibitors bind only to the catalytically active NS2B-NS3pro protease in the closed conformation. ZIKV NS2B-NS3pro was incubated with aprotinin or WRPK3 and the purified complexes were tested in the RNA FP assays. As depicted in FIG. 5B, there was no RNA binding to either of the complexes. Since the active site of closed ZIKV protease is negatively charged (FIG. 2), direct binding of negatively-charged RNA to the same site as aprotinin or WRPK3 is unlikely. The substrate-mimicking inhibitors can eliminate RNA binding by inducing and sustaining the closed conformation of NS2B-NS3pro, which can be incompatible with RNA binding.

### Example 4: Novel Inhibitors of NS2B-NS3pro Can Inhibit RNA Binding and Protease Activity

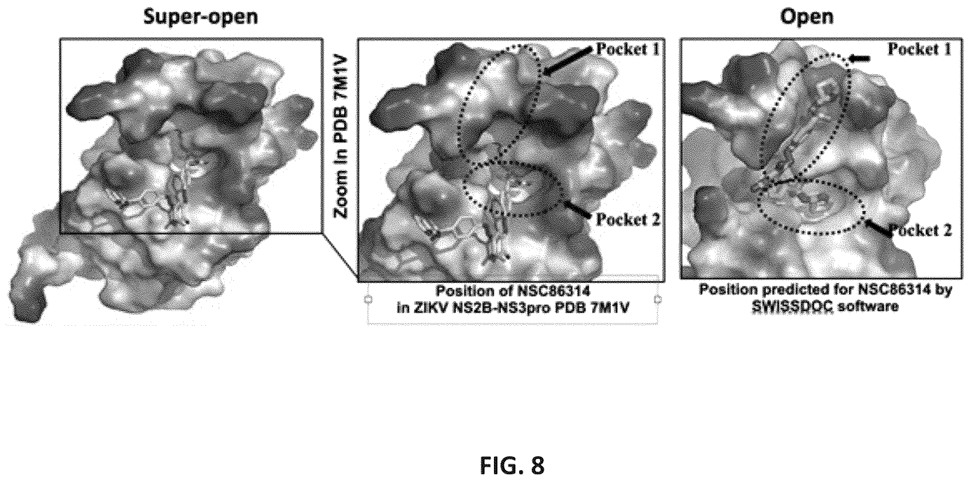

The open conformation of NS2B-NS3pro is formed by rearrangement/dissociation of the NS2B cofactor from the C-terminal half of NS3pro, leading to a loss of proteolytic activity. NS2B dissociation uncovers a hydrophobic cleft in NS3pro, which presents a druggable pocket that is relatively conserved between the flaviviruses. Allosteric inhibitors of ZIKV, WNV, and DENV2 NS2B-NS3pro were designed in silico to bind a region of NS2B-NS3pro distant from the proteolytic active site. These inhibitors showed sub- or low-micromolar IC50 values in vitro for several flaviviral proteases, and had no detectable effect on host serine proteases (furin and other proprotein convertases) with a similar substrate specificity. RNA binding to ZIKV NS2B-NS3pro was tested with a sub-library of these inhibitors, and some compounds were potent inhibitors of RNA binding to ZIKV NS2B-NS3pro, as depicted in FIG. 7. In Top of FIG. 7, small molecule allosteric inhibitors (10 μM) targeting open/super-open conformations of ZIKV NS2B-NS3pro block ssRNA binding. Fluorescent polarization (%) using FAM labeled ssRNA (20 poly-rU). * p<0.05, ** p<0.05, *** p<0.0005, **** p<0.0005 by two-tailed unpaired t-test with Welch's correction. In Bottom of FIG. 7, IC₅₀ for inhibition of ZIKV, WNV, and DENV2 NS2B-NS3pro proteolysis by the indicated inhibitors. One compound, NSC86314, very effectively inhibited binding to NS2B-NS3pro from ZIKV, WNV, and DENV in vitro with low to sub-micromolar IC50 values. NSC86314 was previously shown to inhibit WNV and DENV2 replicons in cell-based assays with IC50 values of<50 μM. A global search of docking locations on the ZIKV NS2B-NS3pro surface using the SWISSDOCK program (www.swissdock.ch) points to novel druggable pockets located outside the active site in the open and super-open conformations as the docking site in both WNV and ZIKV NS2B-NS3pro polypeptides, as depicted in FIG. 8.

To confirm the in silico docking results, NSC86314 was co-crystallized with the Mut7 ZIKV NS2B-NS3pro construct. Two additional mutations (Leu30Thr and Leu31Ser) were introduced into the membrane-binding loop to disrupt/modify the dimer interface of NS2B-NS3 protease. In our previously reported structures of NS2B-NS3pro in the super-open conformations, Leu30 and Lue31 were inserted into the hydrophobic cleft of the C-terminal ß-barrel, preventing binding of inhibitors that target these areas (PDB ID 6UM3). The Mut7 crystal structure was solved and refined to a resolution of 1.8 Å (PDB ID 7MIV). The structure revealed that NSC83614 binds to the predicted hydrophobic pocket 2 near the C-terminus of NS3pro, as shown in FIG. 8). However, in contrast to the docking model, the dimerization of Mut7 and the structural constraints around pocket 1 made it inaccessible for a part of this inhibitor.

In a Virtual Ligand Screening (VLS) of the NCI Chemotherapeutic Agents Repository, NSC114428 was identified to be an inhibitor of both the proteolytic activity of ZIKV protease and RNA binding in the low μM range, as shown in the FIG. 7 bar graph. Additionally, a series of flavivirus replication inhibitors with activity against ZIKV, WNV, and DENV proteases exhibited inhibition of RNA binding to ZIKV NS2B-NS3pro. The most potent inhibitors of ZIKV and DENV2 protease activity tended to also be the most potent inhibitors of RNA binding as shown in FIG. 7. However, this association was not observed for inhibition of WNV protease activity, and the relationship between inhibition of flavivirus proteolytic activity and RNA binding can be complex. These results provide proof-of-principle for a new class of allosteric inhibitors that specifically target newly identified druggable pockets present in the open and super-open conformations of ZIKV NS2B-NS3pro. Such allosteric inhibitors can be able to block both the proteolytic and RNA-binding activities of NS2B-NS3pro from multiple flaviviruses.

### Example 5: Modeling RNA Binding to ZIKV NS2B-NS3 and RNA-Helicase Interactions

The biochemical findings provided evidence that NS2-NS3pro binds RNA thus possibly cooperating with NS3helicase machinery (NS3hel) that unwinds double stranded RNA. Structural modeling proceeded through the following steps: a) building a model of RNA-NS2B-NS3pro complex; b) explore relative position and conformations of the NS2B-NS3pro and NS3hel domains; c) building a model of RNA bound to NS2B-NS3 complex.

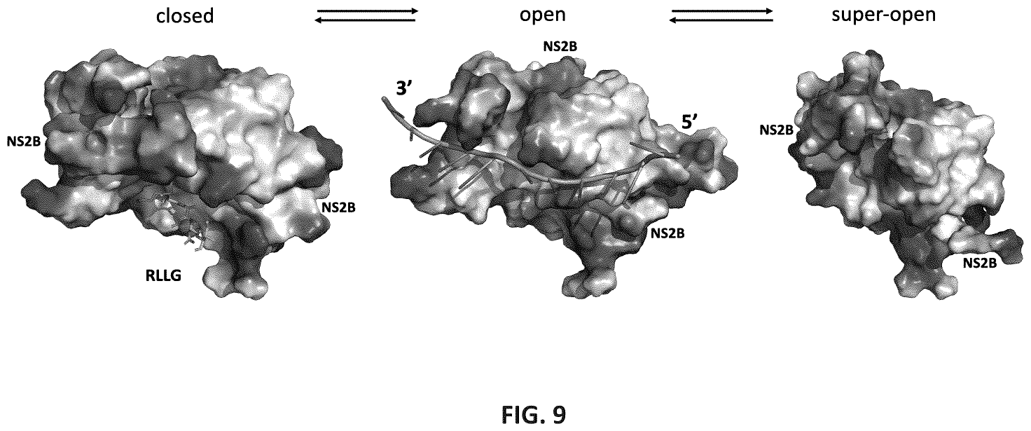

FIG. 9 illustrates the closed (PDB ID: 5LC0), open (model based on WNV PDB ID: 2GGV) and super-open (PDB ID: 7MIV) conformations of ZIKV NS2B-NS3. The open conformation is shown with RNA inserted into the fork-like structures (here referred as forks) composed of positively charged amino acids. One fork is close to 3′-end and another is located in the middle of the RNA strand. Orientation of the enzymes is chosen in such a way that RLLG loop interacting with the membrane surface is facing down. As shown in FIG. 9, these forks were obstructed or misshaped in the closed and super-open conformations. Modeled of RNA binding through the energy minimized using Amber18 and ff14SB force field revealed a plausible position of RNA strand readily accommodated by both forks (FIG. 9).

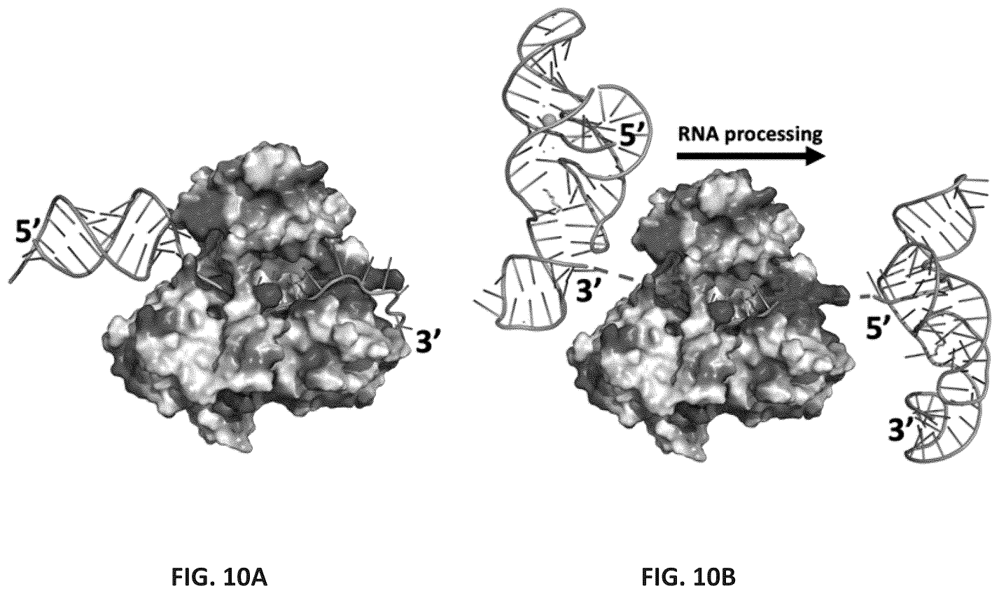

Next single strand RNA interactions with ZIKV NS3hel was modeled. The structure of ZIKV NS3hel bound to a short RNA fragment, AGAUC, (PDB ID: 5GJB) was used to model a longer strand of the RNA by extending the short fragment along with structure optimization. A bacterial helicase bound to a single stranded DNA (PDB ID: 2P6R) was used to build the initial configuration of the ZIKV helicase-RNA complex using 5GJB structure as a template. As depicted in FIG. 10A, this model contained a double helical part of RNA, before splitting and unfolding, oriented toward the 5′-end of the leading strand, and a single stranded RNA extending toward 3′-end. This structure was then energy minimized using Amber18 and ff14SB force field. The ssRNA movement is facilitated by a patch of positively charged residues (right side of NS3hel—blue color). During minimization the positional constraints were applied to the nucleotides, which positions coincide with those observed in the 5GJB crystal structure. As depicted in FIG. 10B, movement of the double stranded RNA is further guided by interactions with positively charged groove (blue color) located on the right side of the NS3hel. FIG. 10B illustrates the crystal structure (PDB id: 5GJB) of ZIKV NS3hel with short fragment of ssRNA (orange) and tentative positions of ZIKV RNA knots (PDB id: 5TPY), which explains why RNA is processed from 3′-end toward 5′-end. Unwinding the knot is much more difficult if it is done via pulling its 5′-end (right side knot), due to stronger interaction with other nucleotides, then via pulling with its 3′-end (left side knot). A uniquely stable multi-pseudoknot structure for ZIKV RNA was used to evaluate the direction of RNA processing. The crystal structure of NS3hel with fragment of RNA (PDB ID: 5GJB) was combined with tentative positions of RNA knots (PDB ID: 5TPY), shown in FIG. 10B. When the RNA knot is located on the left side of NS3hel, i.e. it is attached via its 3′-end to the RNA-NS3hel complex, then the RNA processing involves unconstrained unwinding of the knot. In the case when RNA knot is on the right side of NS3hel, attached via 5′-end to RNA-NS3hel complex, the knot cannot be easily unfolded due to strong interactions of its 5′-end with the remaining part of the RNA knot. This modeling supports a scenario where the short RNA fragment, AGAUC, elongated from the 3′-end to fill the positively charged groove represents the nascent single strand RNA threaded through NS3hel (FIG. 10B).

### Example 6: Modeling of NS3-pro-NS3-help Structure, Comparative Analysis of RNA Binding to NS2B-NS3 Conformations, and a “Reverse Inchworm” Model

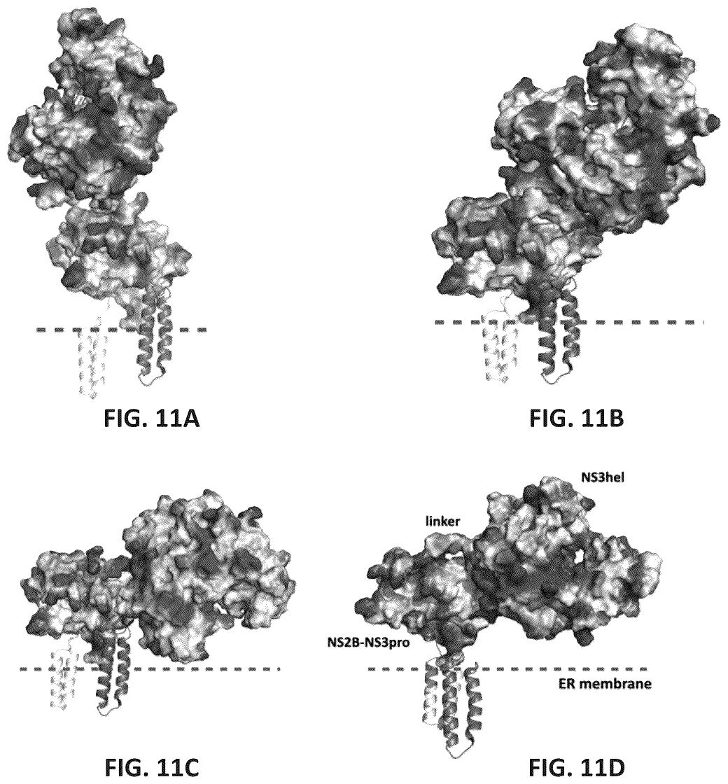

To explore the flexibility of the linker between NS3pro and NS3hel domains possible mutual orientations of these domains were modeled, focusing on NS2B-NS3pro in the open conformation and NS3hel built using three different templates. Because the structure of ZIKV NS2B-NS3pro-NS3hel complex (i.e. protease and helicase together) is not available, three crystallographic structures were obtained from homologous MVE (PDB ID: 2WV9) and DENV4 (PDB IDs: 2WZQ, 2WHX) flaviviruses representing different mutual orientations of NS3pro and NS3hel domains. These available structures demonstrate ample flexibility of the linker connecting the two domains. ZIKV NS3pro-NS3hel was built by structural alignment of ZIKV NS3hel (PDB ID: 5GJB) and the structures of NS2B-NS3pro was built in closed (PDB ID: 5lc0), open (our own model) and super-open (PDB ID: 7m1v) conformations modeled onto MVE and DENV4 crystal structures. The results of modeling illustrate wide range of motion available for NS3hel with respect to NS2B-NS3pro, as depicted in FIGS. 11A-11D. The exact position of NS3hel could be adjusted in all models via rotation that involve only the movements of the flexible linker between NS3pro and NS3hel to arrive at the juxtaposition of the patch of positively charged residues in NS3hel domain and the positively charged forks in the open conformation of NS2B-NS3pro (FIG. 11D). FIG. 11A depicts the 2WV9 structure template from MEV virus. FIG. 11B depicts the 2WHX structure template from DENV4; FIG. 11C depicts the 2WZQ structure template from DENV4. FIG. 11D depicts a homology-built model oriented to juxtapose the patch of positively charged residues in NS3hel domain and the positively charged forks in the open conformation of NS2B-NS3pro. The N- and C-terminal helices of NS2B (grey and sky-blue ribbons, respectively) are oriented to be inserted in the ER membrane.

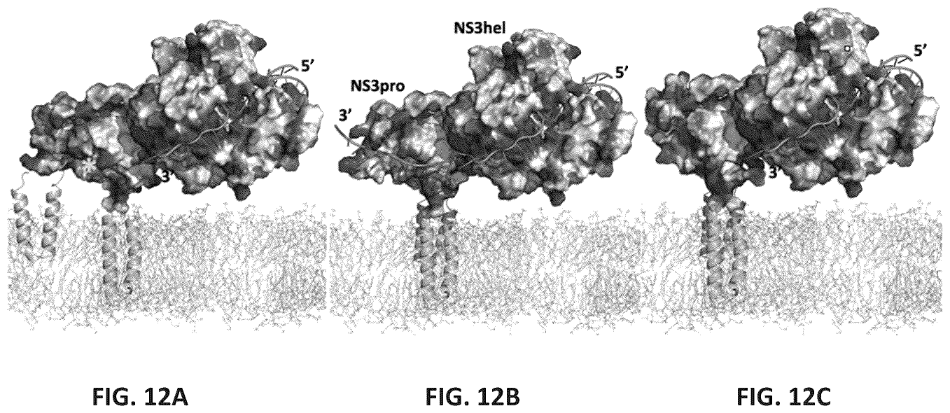

The juxtaposed model depicted in FIG. 11D offers an appealing possibility of RNA binding along the positively charges grove on NS3hel and the forks on NS3pro. This model was used to fit an extended strand of RNA by performing several steps of energy minimized with Amber18 and ff14SB force field resulting in a model of RNA-NS2B-NS3pro-NS3hel where the NS2B-NS3pro is in the open conformation (FIG. 12B). To provide a more realistic view of the complex the N- and C-terminal helices of NS2B were oriented to be inserted into the ER membrane that was modeled to scale using a fragment of the membrane (PDB id: 2MLR) (FIGS. 12A-12C). Similar orientations of NS2B-NS3pro with respect to NS3hel in (FIG. 12A) closed (PDB ID: 5LC0), (FIG. 12B) open (model based on Denv4 PDB ID: 2GGV), and (FIG. 12C) super-open (PDB ID: 7MIV) conformations. Position of the protease active site of NS2B-NS3pro in closed conformation is marked by a yellow asterisk. RNA strand in FIG. 12B is modeled to span a negatively charged surface of NS3hel contiguous with negatively charged forks in the open conformation of NS2B-NS3pro. Shorter RNA strands associated only with NS3hel domain is shown in FIG. 12A and FIG. 12C reflecting the lack of apparent RNA binding structures in the closed and super-open conformations of NS2B-NS3. Only in the open conformation the two fork patches of positively charged amino acids provided a plausible binding for the RNA strand. In the closed conformation the RNA binding volume is occupied by negatively charges NS2B cofactor obstructing RNA binding. In the super-open conformations, the positively charged forks could not be readily observed.

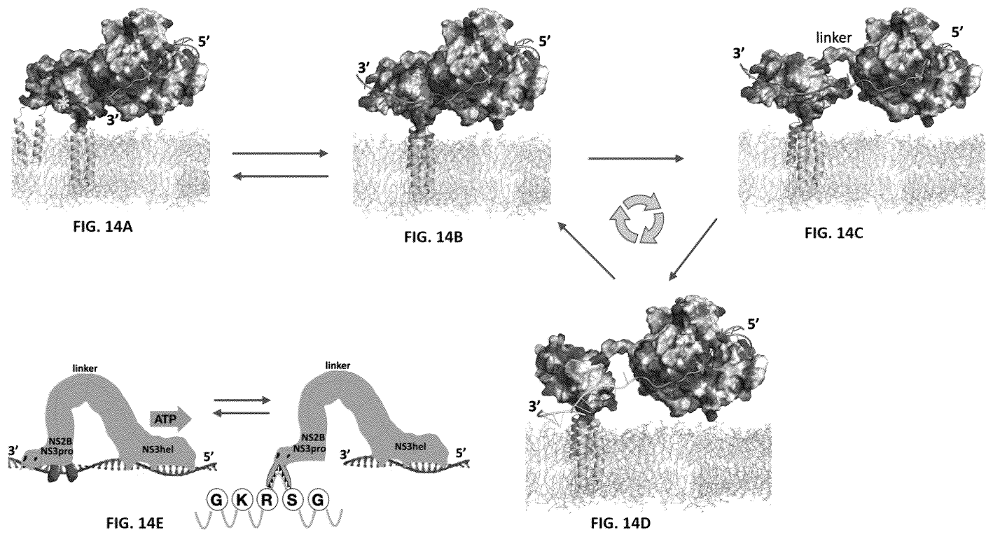

A novel model for a tightly intertwined NS2B-NS3 helicase-protease machinery of ZIKV can thus be proposed, which could be readily extended to all flaviviruses given a very high degree of sequence and structure conservation of their NS2B-NS3 complex (FIGS. 14A-14E). According to this model, a positively charged groove on the NS3hel surface accommodates the threaded single strand RNA and “hands it over” to NS2B-NS3pro. The open conformation of NS2B-NS3pro provides two positively charged/polar forks, contiguous with positively charged groove on NS3hel; these forks bind single strand RNA threaded by NH3hel (FIG. 14B). The ATP-driven NS3hel continues unwinding the double strand RNA and threading the single strand RNA. Because NS2B-NS3Pro is bound to RNA, the NS3hel processivity results in the elongation of the linker between NS3pro and NS3hel domains (FIG. 14B). At some point the linker is elongated to the maximum exerting a pull or “yank” on NS3pro that results in a change of NS2B-NS3pro conformation to the super-open and a release of the bound RNA strand (FIG. 14D). The NS2B-NS3pro domain is then relaxes back to the open conformation adjacent to NH3hel to enter the new cycle of RNA binding. This dynamic cycle of RNA binding and releasing enables the unlimited helicase processivity along the ˜11 kb of dsRNA flavivirus genome. The protease activity can require a distinct closed conformation of NS2B-NS3, which can be induced by the presence of a substrate such as peptide loops extended from the ER membrane (FIG. 14A).

### Example 7: Drug Design Using A Novel Approach: Targeting NS3pro Functions of Flaviviruses

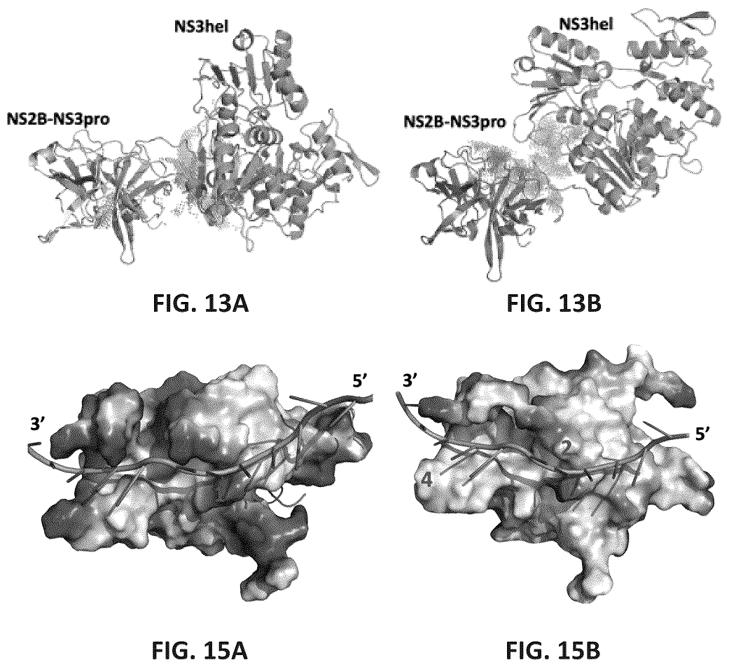

A particular spatial arrangement of NS2B-NS3pro and NS3hel can enable RNA binding to both domains simultaneously via negatively charged elements, which can be an important element in viral replication cycle. Our model has demonstrated that targeting such specific alignment with small molecules (e.g., a dual binding molecules that bridge both domains) can be a promising therapeutic strategy. Such small molecules could be fit to the cavities located at the interface between NS3pro and NS3hel. We thus evaluated the sizes and locations of these cavities on the interface of ZIKV NS2B-NS3pro and NS3hel with the POVME3.0 software. FIG. 13A illustrates the positions of the cavities at the interface of NS3pro and NS3hel in the structure built on rotated 2WHX as a template. FIG. 13B illustrates the cavities at the interface of NS3pro and NS3hel in the structure built using original 2WHX structure as a template. The colored meshed areas mark the positions of cavities. The volume measurements are in the range of 150-680 A³ for NS3hel and 300-650 A³ for NS3pro, thus a combined volume of 500-1300 A³ is available to accommodate a dual binding molecule (FIG. 10). For comparison, the volume of ATP molecule is ˜650 A³. Therefore, both individual and combined volumes of such cavities could accommodate small molecules that can be designed to interfere with this specific alignment of NS2B-NS3pro and NS3hel.

The amino acid sequences underlying RNA-binding forks are conserved across flaviviruses, as illustrated in FIG. 10A of the biorxiv article updated version, indicating their functional importance for RNA-binding. The alignment of NS3pro from 11 flaviviruses, shown in FIG. 10A of the biorxiv article, revealed a near perfect identify/charge conservation for the forks 1-3 and to lesser degree for the 4th fork. Note that although not positively charged, Asparagine (N) and Glutamine (Q) side chains emphasized in 4 locations of the alignment are polar, and are known to readily form hydrogen bonds potentially stabilizing RNA strand. Numbers 1-4 in red in FIG. 10A of the biorxiv article updated version and FIGS. 15A-15B indicate corresponding conserved amino acids/structural positions of RNA-binding forks. As depicted in FIGS. 15A and 15B, modeling of RNA fit with open conformations of ZIKV NS2B-NS3pro and WNV NS2B-NS3pro (PDB ID: 2GGV) suggested that open conformation protease domains of both viruses are well posed to bind RNA. Chemical or biological entities, such as small molecules or antibodies, which can interfere with the RNA-binding forks of NS2B-NS3pro within the alignment of NS2B-NS3pro and NS3hel can be designed, screened, identified, and developed for their potential antiviral activities against flaviviruses.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A method of inhibiting replication of a flavivirus comprising a NS2B-NS3pro domain and a NS3hel domain, comprising: administering a chemical entity that interacts with the NS2B-NS3pro domain in an open conformation and the NS3hel domain.

2. The method of claim 1, wherein the chemical entity blocks a single strand RNA from interacting with the NS2B-NS3pro domain and the NS3hel domain.

3. The method of claim 1, wherein the chemical entity comprises a NS2B-NS3pro binding moiety and a NS3hel binding moiety.

4. The method of claim 3, wherein the chemical entity is a small molecule, a modified single strand RNA, a polynucleotide, a modified polypeptide, a fusion protein or an antibody.

5. The method of claim 4, wherein the chemical entity is a small molecule.

6. The method of claim 5, wherein the NS2B-NS3pro binding moiety and the NS3hel binding moiety of the small molecule are connected by a linker.

7. The method of claim 1, wherein the chemical entity interacts with a positively charged groove on a surface of the NS3hel domain and two positively charged/polar forks of the NS2B-NS3pro domain.

8. The method of claim 1, wherein the chemical entity is an antibody.

9. The method of claim 8, wherein the antibody is a human antibody or humanized antibody.

10. The method of claim 1, wherein the chemical entity is a fusion protein.

11. The method of claim 10, wherein the fusion protein comprises human or humanized regions.

12. The method of claim 1, wherein the flavivirus is Zika virus (ZIKV), West Nile virus (WNV), dengue virus (DENV serotypes 1-4), Japanese encephalitis virus, hepatitis C virus (HCV), or tick-borne encephalitis virus.

13. A method of inhibiting replication of a flavivirus, comprising: administering a chemical entity or a biological entity that simultaneously interferes with single strand RNA binding and protease catalytic activity during viral propagation of the flavivirus.

14. The method of claim 13, wherein the chemical entity or the biological entity is an anti-NS2B-NS3pro inhibitor.

15. The method of claim 14, wherein the anti-NS2B-NS3pro inhibitor targets one or more pockets of a NS2B-NS3pro domain when the NS2B-NS3pro domain is in an open and/or super-open conformation.

16. The method of claim 14, wherein the anti-NS2B-NS3pro inhibitor targets the cavities formed by the closely opposed ZIKV NS3pro and NS3hel domains thus inhibiting the alignment of the NS3pro and NS3hel domains.

17. The method of claim 13, wherein the flavivirus is Zika virus (ZIKV).
